(19) United States
(12) Patent Application Publication
(10) Pub. No.: US 2008/0167322 A1
(43) Pub. Date: Jul. 10, 2008

(54) Substituted Pyrazoles as Modulators of Chemokine Receptors

(76) Inventors: Anthony B. Pinkerton, San Diego, CA (US); Rowena Cube, Harleysville, PA (US); John Hutchinson, La Jolla, CA (US); Dehua Huang, San Diego, CA (US); Jean-Michel Vernier, San Diego, CA (US); Dong-Ming Shen, Edison, NJ (US)

Correspondence Address: MERCK AND CO., INC, P O BOX 2000, RAHWAY, NJ, 07065-0907, US

(21) Appl. No.: 11/884,325

(22) PCT Filed: Feb. 14, 2006
(86) PCT No.: PCT/US06/05075

§ 371 Date: Aug. 13, 2007

Related U.S. Application Data
(60) Provisional application No. 60/653,326, filed on Feb. 16, 2005.
(60) Provisional application No. 60/660,364, filed on Mar. 10, 2005.

(51) Int. Cl. A61K 31/497; C07D 231/10; A61K 31/4155; A61P 19/02; C07D 401/02; A61K 31/4439

(52) U.S. Cl. 514/255.05; 548/370.1; 514/407; 514/341; 546/276.1; 544/405

(57) ABSTRACT

Substituted pyrazole compounds such compounds represented by formula I: which are used to modulate the CCR-2 chemokine receptor to prevent or treat inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis; and pharmaceutical compositions comprising these compounds and the use of these compounds and compositions.

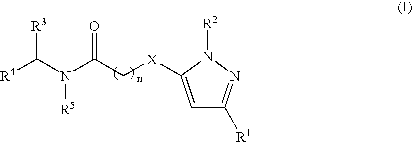

### BACKGROUND OF THE INVENTION

The present invention is directed to substituted pyrazole compounds useful as modulators of chemokine receptors.

The chemokines are a family of small (70-120 amino acids), proinflammatory cytokines, with potent chemotactic activities. Chemokines are chemotactic cytokines that are released by a wide variety of cells to attract various cells, such as monocytes, macrophages, T cells, eosinophils, basophils and neutrophils to sites of inflammation (reviewed in Schall, Cytokine, 3, 165-183 (1991) and Murphy, Rev. Immun., 12, 593-633 (1994)). These molecules were originally defined by four conserved cysteines and divided into two subfamilies based on the arrangement of the first cysteine pair. In the CXC-chemokine family, which includes IL-8, GROα, NAP-2 and EP-10, these two cysteines are separated by a single amino acid, while in the CC-chemokine family, which includes RANTES, MCP-1, MCP-2, MCP-3, MIP-1α, MIP-1β and eotaxin, these two residues are adjacent.

The α-chemokines, such as interleukin-8 (IL-8), neutrophil-activating protein-2 (NAP-2) and melanoma growth stimulatory activity protein (MGSA) are chemotactic primarily for neutrophils, whereas β-chemokines, such as RANTES, MIP-1α: MIP-1β, monocyte chemotactic protein-1 (MCP-1), MCP-2, MCP-3 and eotaxin are chemotactic for macrophages, monocytes, T-cells, eosinophils and basophils (Deng, et al., Nature, 381, 661-666 (1996)).

The chemokines are secreted by a wide variety of cell types and bind to specific G-protein coupled receptors (GPCRs) (reviewed in Horuk, Trends Pharm. Sci., 15, 159-165 (1994)) present on leukocytes and other cells. These chemokine receptors form a sub-family of GPCRs, which, at present, consists of fifteen characterized members and a number of orphans. Unlike receptors for promiscuous chemoattractants such as C5a, fMLP, PAF, and LTB4, chemokine receptors are more selectively expressed on subsets of leukocytes. Thus, generation of specific chemokines provides a mechanism for recruitment of particular leukocyte subsets.

On binding their cognate ligands, chemokine receptors transduce an intracellular signal though the associated trimeric G protein, resulting in a rapid increase in intracellular calcium concentration. There are at least seven human chemokine receptors that bind or respond to β-chemokines with the following characteristic pattern: CCR-1 (or “CKR-1” or “CC-CKR-1”) [MIP-1α, MIP-1β, MCP-3, RANTES] (Ben-Barruch, et al., J. Biol. Chem., 270, 22123-22128 (1995); Beote, et al, Cell 72, 415-425 (1993)); CCR-2A and CCR-2B (or “CKR-2A”/“CKR-2A” or “CC-CKR-2A”/“CC-CKR-2A”) [MCP-1, MCP-2, MCP-3, MCP-4]; CCR-3 (or “CKR-3” or “CC-CKR-3”) [Eotaxin, Eotaxin 2, RANTES, MCP-2, MCP-3] (Rollins, et al., Blood, 90, 908-928 (1997)); CCR-4 (or “CKR-4” or “CC-CKR-4”) [MIP-1α, RANTES, MCP-1] (Rollins, et al., Blood, 90, 908-928 (1997)); CCR-5 (or “CKR-5” or “CC-CKR-5”) [MIP-1α, RANTES, MIP-1β] (Sanson, et al., Biochemistry, 35, 3362-3367 (1996)); and the Duffy blood-group antigen [RANTES, MCP-1] (Chaudhun, et al., J. Biol. Chem., 269, 7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”) among other chemokines.

Chemokine receptors, such as CCR-1, CCR-2, CCR-2A, CCR-2B, CCR-3, CCR-4, CCR-5, CXCR-3, CXCR-4, have been implicated as being important mediators of inflammatory and immunoregulatory disorders and diseases, including asthma, rhinitis and allergic diseases, as well as autoimmune pathologies such as rheumatoid arthritis and atherosclerosis. Humans who are homozygous for the 32-basepair deletion in the CCR-5 gene appear to have less susceptibility to rheumatoid arthritis (Gomez, et al., Arthritis & Rheumatism, 42, 989-992 (1999)). A review of the role of eosinophils in allergic inflammation is provided by Kita, H., et al., J. Exp. Med. 183, 2421-2426 (1996). A general review of the role of chemokines in allergic inflammation is provided by Lustger, A. D., New England J. Med., 338(7), 426-445 (1998).

A subset of chemokines are potent chemoattractants for monocytes and macrophages. The best characterized of these is MCP-1 (monocyte chemoattractant protein-1), whose primary receptor is CCR2. MCP-1 is produced in a variety of cell types in response to inflammatory stimuli in various species, including rodents and humans, and stimulates chemotaxis in monocytes and a subset of lymphocytes. In particular, MCP-1 production correlates with monocyte and macrophage infiltration at inflammatory sites. Deletion of either MCP-1 or CCR2 by homologous recombination in mice results in marked attenuation of monocyte recruitment in response to thioglycollate injection and Listeria monocytogenes infection (Lu et al., J. Exp. Med., 187, 601-608 (1998); Kurihara et al. J. Exp. Med., 186, 1757-1762 (1997); Boring et al. J. Clin. Invest., 100, 2552-2561 (1997); Kuziel et al. Proc. Natl. Acad. Sci., 94, 12053-12058 (1997)). Furthermore, these animals show reduced monocyte infiltration into granulomatous lesions induced by the injection of schistosomal or mycobacterial antigens (Boring et al. J. Clin. Invest., 100, 2552-2561 (1997); Warmington et al. Am J. Path., 154, 1407-1416 (1999)). These data suggest that MCP-1-induced CCR2 activation plays a major role in monocyte recruitment to inflammatory sites, and that antagonism of this activity will produce a sufficient suppression of the immune response to produce therapeutic benefits in immunoinflammatory and autoimmune diseases.

Accordingly, agents which modulate chemokine receptors such as the CCR-2 receptor would be useful in such disorders and diseases.

In addition, the recruitment of monocytes to inflammatory lesions in the vascular wall is a major component of the pathogenesis of atherogenic plaque formation. MCP-1 is produced and secreted by endothelial cells and intimal smooth muscle cells after injury to the vascular wall in hypercholesterolemic conditions. Monocytes recruited to the site of injury infiltrate the vascular wall and differentiate to foam cells in response to the released MCP-1. Several groups have now demonstrated that aortic lesion size, macrophage content and necrosis are attenuated in MCP-1 −/− or CCR2 −/− mice backcrossed to APO-E −/−, LDL-R −/− or Apo B transgenic mice maintained on high fat diets (Boring et al. Nature, 394, 894-897 (1998); Gosling et al. J. Clin. Invest., 103, 773-778 (1999)). Thus, CCR2 antagonists may inhibit atherosclerotic lesion formation and pathological progression by impairing monocyte recruitment and differentiation in the arterial wall.

### SUMMARY OF THE INVENTION

The present invention is directed to substituted pyrazole compounds such compounds represented by formula I:

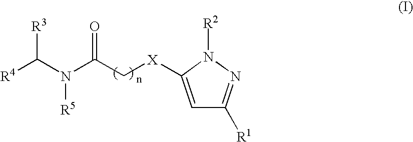

(wherein R¹, R², R³, R⁴, R⁵, n and X are described herein). These compounds are useful as modulators of the CCR-2 chemokine receptor. The present invention is further directed to compounds which are modulators of chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

### DETAILED DESCRIPTION OF THE INVENTION

The present invention is directed to compounds represented by formula (I):

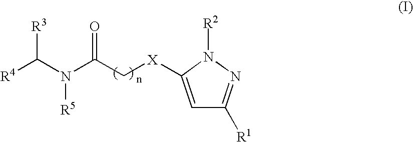

wherein:
R¹ and R² are each independently selected from —(C₀₋₆alkyl)-W—(C₆₋₁₄aryl), —(C₀₋₆alkyl)-W-heterocycle and —(C₀₋₆alkyl)-W—(C₃₋₇cycloalkyl);
R³ and R⁴ are each independently selected from —C₀₋₆alkyl, —(C₀₋₆alkyl)-W—(C₁₋₆alkyl), —(C₀₋₆alkyl)-W—(C₃₋₇cycloalkyl), —(C₀₋₆alkyl)-W—(C₆₋₁₄aryl), C₀₋₆alkyl)-W-heterocycle, and —C(O)OR⁶;
where each of R¹, R², R³ and R⁴ is independently unsubstituted or substituted with 1-7 substituents, where each of said 1-7 substituents is independently selected from halo, hydroxy, —O—C₁₋₃ alkyl, trifluoromethyl, —C₁₋₃ alkyl, —CO₂R⁶, —CN, —N(R⁶)₂, —NR⁶COR⁶, —NRSO₂R⁶, and —CONR⁶, —(C₀₋₆alkyl)-W—R⁶;
   R⁵ is selected from hydrogen, —C₀₋₆alkyl, —(C₀₋₆alkyl)—(C₆₋₁₄aryl), —(C₀₋₆alkyl)-heterocycle, —(C₀₋₆alkyl)-C₃₋₇cycloalkyl and —(C₀₋₆alkyl)-CO₂R⁶;
   R⁶ is independently selected from C₁₋₆alkyl and NR⁵C(N)NH₂, or two R⁶ join to for a ring selected from pyrrolidinyl, piperidinyl and azepanyl;

### X is CH₂, N, O or S;

W is selected from a single bond, —O—, —S—, —SO—, —SO₂—, —CO—, —CO₂—, —CONR⁶— and —NR⁶—;
n is 0-6;
and pharmaceutically acceptable salts and an individual diastereomers thereof.

More particularly, compounds of the present invention also include those of Formula (I)

wherein:
R¹ and R² are each independently selected from —(C₆₋₁₄aryl) and —(C₆₋₁₄heteroaryl);
R³ and R⁴ are each independently selected from —C₀₋₆alkyl, —(C₀₋₆alkyl)—(C₆₋₁₄aryl), —(C₀₋₆alkyl)-(C₆₋₁₄heteroaryl),
where each of R¹, R², R³ and R⁴ is independently unsubstituted or substituted with 1-7 substituents, where each of said 1-7 substituents is independently selected from halo, hydroxy, —O—C₁₋₃ alkyl, trifluoromethyl, —C₁₋₃ alkyl, —CO₂R⁶, —CN, —N(R⁶)₂, —NR⁶COR⁶, —NRSO₂R⁶, and —CONR⁶;
R⁵ is —C₀₋₆alkyl;
R⁶ is independently selected from C₁₋₆alkyl and NR⁵C(N)NH₂, or two R⁶ join to for a ring selected from pyrrolidinyl, piperidinyl and azepanyl;

### X is CH₂ or 0;

W is selected from a single bond, —O—, —S—, —SO—, —SO₂—, —CO—, —CO₂—, —CONR⁶— and —NR⁶—;
n is 0-6;
and pharmaceutically acceptable salts and an individual diastereomers thereof.

Representative compounds of the present invention include those presented in the EXAMPLES and pharmaceutically acceptable salts and individual diastereomers thereof.

The compounds of the instant invention may have an asymmetric center at the carbon bearing groups R³ and R⁴. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the scope of this invention.

The independent syntheses of diastereomers and enantiomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

The term “alkyl” means linear or branched structures and combinations thereof, having the indicated number of carbon atoms. C₀₋₆alkyl refers to a group as having 0, 1, 2, 3, 4, 5 or 6 carbons in a linear or branched arrangement, and so on with respect to other numerical designations. C₀, as in C₀alkyl is a direct covalent bond when in a bridging position and is a hydrogen when in a terminal position. C₁₋₆alkyl includes methyl, ethyl, propyl, 2-propyl, s- and t-butyl, butyl, pentyl, hexyl, 1,1-dimethylethyl, cyclopropyl, cyclobutyl, cyclopentyl and cyclohexyl.

The term “aryl” is intended to mean any stable monocyclic or bicyclic carbon ring of up to 7 members in each ring, wherein at least one ring is aromatic. Examples of such aryl elements include phenyl, napthyl, tetrahydronapthyl, indanyl, or biphenyl.

The term “heteroaryl”, as used herein except where noted, represents a stable 5- to 7-membered monocyclic- or stable 9- to 10-membered fused bicyclic heterocyclic ring system which contains an aromatic ring, any ring of which may be saturated, such as piperidinyl, partially saturated, or unsaturated, such as pyridinyl, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, benzimidazole, benzisothiazole, benzisoxazole, benzo furan, benzothiazole, benzothiophene, benzotriazole, benzoxazole, carboline, cinnoline, furan, furazan, imidazole, indazole, indole, indolizine, isoquinoline, isothiazole, isoxazole, naphthyridine, oxadiazole, oxazole, phthalazine, pteridine, purine, pyran, pyrazine, pyrazole, pyridazine, pyridine, pyrimidine, pyrrole, quinazoline, quinoline, quinoxaline, tetrazole, thiadiazole, thiazole, thiophene, triazine, triazole, and N-oxides thereof.

The term “cycloalkyl” means mono-, bi- or tri-cyclic structures, optionally combined with linear or branched structures, the indicated number of carbon atoms. Examples of cycloalkyl groups include cyclopropyl, cyclopentyl, cycloheptyl, adamantyl, cyclododecylmethyl, and the like.

The term “heterocycle” as used herein is intended to include the following groups: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof.

The term “substituted” or “substituent” in reference to substitution on alkyl, cycloalkyl, phenyl, heterocycle, or some other chemical group is intended to include mono- and poly-substitution by a named substituent to the extent such single and multiple substitution is chemically allowed in any of the named chemical groups. It is understood that the definition of a substituent at a particular location in a molecule is independent of its definition at other locations in the molecule. Thus, for example, when R⁴ is defined as —CONR¹⁰R¹⁰ each R¹⁰ is independently selected from the possible values thereof; i.e., each R¹⁰ can be the same as or different from any other R¹⁰.

The term “optionally substituted” is intended to include both substituted and unsubstituted. Thus, for example, optionally substituted alkyl, where halo was an optional substituent, could represent a propyl or fluoro-propyl.

As appreciated by those of skill in the art, halo or halogen as used herein are intended to include chloro, fluoro, bromo and iodo.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be prepared from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile are used. Suitable salts are found, e.g. in Remington's Pharmaceutical Sciences, 17th ed., Mack Publishing Company, Easton, Pa., 1985, p. 1418.

Exemplifying the invention is the use of the compounds disclosed in the Examples and herein.

Specific compounds within the present invention include a compound which selected from the group consisting of: the title compounds of the Examples;

and pharmaceutically acceptable salts thereof and individual diastereomers thereof.

The subject compounds are useful in a method of modulating chemokine receptor activity in a patient in need of such modulation comprising the administration of an effective amount of the compound.

The present invention is directed to the use of the foregoing compounds as modulators of chemokine receptor activity. In particular, these compounds are useful as modulators of the chemokine receptors, in particular CCR-2.

The utility of the compounds in accordance with the present invention as modulators of chemokine receptor activity may be demonstrated by methodology known in the art, such as the assay for chemokine binding as disclosed by Van Riper, et al., J. Exp. Med., 177, 851-856 (1993) which may be readily adapted for measurement of CCR-2 binding.

Receptor affinity in a CCR-2 binding assay was determined by measuring inhibition of ¹²⁵I-MCP-1 to the endogenous CCR-2 receptor on various cell types including monocytes, THP-1 cells, or after heterologous expression of the cloned receptor in eukaryotic cells. The cells were suspended in binding buffer (50 mM HEPES, pH 7.2, 5 mM MgCl₂, 1 mM CaCl₂, and 0.50% BSA or 0.5% human serum) and added to test compound or DMSO and ¹²⁵I-MCP-1 at room temperature for 1 h to allow binding. The cells were then collected on GFB filters, washed with 25 mM HEPES buffer containing 500 mM NaCl and cell bound ¹²⁵I-MCP-1 was quantified.

In a chemotaxis assay chemotaxis was performed using T cell depleted PBMC isolated from venous whole or leukophoresed blood and purified by Ficoll-Hypaque centrifugation followed by resetting with neuraminidase-treated sheep erythrocytes. Once isolated, the cells were washed with HBSS containing 0.1 mg/ml BSA and suspended at 1×10⁷ cells/ml. Cells were fluorescently labeled in the dark with 2 μM Calcien-AM (Molecular Probes), for 30 min at 37° C. Labeled cells were washed twice and suspended at 5×10⁶ cells/ml in RPMI 1640 with L-glutamine (without phenol red) containing 0.1 mg/ml BSA. MCP-1 (Peprotech) at 10 ng/ml diluted in same medium or medium alone were added to the bottom wells (27 μl). Monocytes (150,000 cells) were added to the topside of the filter (30 μl) following a 15 min preincubation with DMSO or with various concentrations of test compound. An equal concentration of test compound or DMSO was added to the bottom well to prevent dilution by diffusion. Following a 60 min incubation at 37° C., 5% CO₂, the filter was removed and the topside was washed with HBSS containing 0.1 mg/ml BSA to remove cells that had not migrated into the filter. Spontaneous migration (chemokinesis) was determined in the absence of chemoattractant

In particular, the compounds of the following examples had activity in binding to the CCR-2 receptor in the aforementioned assays, generally with an IC₅₀ of less than about 1 μM. Such a result is indicative of the intrinsic activity of the compounds in use as modulators of chemokine receptor activity.

Mammalian chemokine receptors provide a target for interfering with or promoting eosinophil and/or leukocyte function in a mammal, such as a human. Compounds which inhibit or promote chemokine receptor function, are particularly useful for modulating eosinophil and/or leukocyte function for therapeutic purposes. Accordingly, compounds which inhibit or promote chemokine receptor function would be useful in treating, preventing, ameliorating, controlling or reducing the risk of a wide variety of inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis, and further, chronic obstructive pulmonary disease, and multiple sclerosis.

For example, an instant compound which inhibits one or more functions of a mammalian chemokine receptor (e.g., a human chemokine receptor) may be administered to inhibit (i.e., reduce or prevent) inflammation. As a result, one or more inflammatory processes, such as leukocyte emigration, chemotaxis, exocytosis (e.g., of enzymes, histamine) or inflammatory mediator release, is inhibited.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases and conditions associated with inflammation and infection can be treated using the compounds of the present invention. In a certain embodiment, the disease or condition is one in which the actions of leukocytes are to be inhibited or promoted, in order to modulate the inflammatory response.

Diseases or conditions of humans or other species which can be treated with inhibitors of chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersentitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinophilic myositis, eosinophilic fasciitis; and cancers, including cancers with leukocyte infiltration of the skin or organs and other cancers. Inhibitors of chemokine receptor function may also be useful in the treatment and prevention of stroke (Hughes et al., Journal of Cerebral Blood Flow & Metabolism, 22:308-317, 2002, and Takami et al., Journal of Cerebral Blood Flow & Metabolism, 22:780-784, 2002), neurodegenerative conditions including but not limited to Alzheimer's disease, amyotrophic lateral sclerosis (ALS) and Parkinson's disease, obesity, type II diabetes, metabolic syndrome, neuropathic and inflammatory pain, and Guillain Barre syndrome. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis, fibrosis, and chronic obstructive pulmonary disease.

Diseases or conditions of humans or other species, which can be treated with modulators of chemokine receptor function, include or involve but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum).

In addition, treatment of the aforementioned inflammatory, allergic, infectious and autoimmune diseases can also be contemplated for agonists of chemokine receptor function if one contemplates the delivery of sufficient compound to cause the loss of receptor expression on cells through the induction of chemokine receptor internalization or delivery of compound in a manner that results in the misdirection of the migration of cells.

The compounds of the present invention are accordingly useful in treating, preventing, ameliorating, controlling or reducing the risk of a wide variety of inflammatory and immunoregulatory disorders and diseases, allergic conditions, atopic conditions, as well as autoimmune pathologies. In a specific embodiment, the present invention is directed to the use of the subject compounds for treating, preventing, ameliorating, controlling or reducing the risk of autoimmune diseases, such as rheumatoid arthritis, psoriatic arthritis and multiple sclerosis.

In another aspect, the instant invention may be used to evaluate putative specific agonists or antagonists of chemokine receptors, including CCR-2. Accordingly, the present invention is directed to the use of these compounds in the preparation and execution of screening assays for compounds that modulate the activity of chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CCR-2. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for modulating chemokine receptor activity, in humans and animals comprising combining a compound of the present invention with a pharmaceutical carrier or diluent.

The present invention is further directed to the use of the present compounds in treating, preventing, ameliorating, controlling or reducing the risk of infection by a retrovirus, in particular, herpes virus or the human immunodeficiency virus (HIV) and the treatment of, and delaying of the onset of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by, e.g., blood transfusion, organ transplant, exchange of body fluids, bites, accidental needle stick, or exposure to patient blood during surgery.

In a further aspect of the present invention, a subject compound may be used in a method of inhibiting the binding of a chemokine to a chemokine receptor, such as CCR-2, of a target cell, which comprises contacting the target cell with an amount of the compound which is effective at inhibiting the binding of the chemokine to the chemokine receptor.

The subject treated in the methods above is a mammal, for instance a human being, male or female, in whom modulation of chemokine receptor activity is desired. “Modulation” as used herein is intended to encompass antagonism, agonism, partial antagonism, inverse agonism and/or partial agonism. In an aspect of the present invention, modulation refers to antagonism of chemokine receptor activity. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

As used herein, the term “treatment” refers both to the treatment and to the prevention or prophylactic therapy of the aforementioned conditions.

Combined therapy to modulate chemokine receptor activity for thereby treating, preventing, ameliorating, controlling or reducing the risk of inflammatory and immunoregulatory disorders and diseases, including asthma and allergic diseases, as well as autoimmune pathologies such as rheumatoid arthritis and multiple sclerosis, and those pathologies noted above is illustrated by the combination of the compounds of this invention and other compounds which are known for such utilities.

For example, in treating, preventing, ameliorating, controlling or reducing the risk of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, biological TNF sequestrants, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention may be used. Accordingly, the pharmaceutical compositions of the present invention include those that also contain one or more other active ingredients, in addition to a compound of the present invention.

Examples of other active ingredients that may be combined with CCR2 antagonists, such as the CCR2 antagonists compounds of the present invention, either administered separately or in the same pharmaceutical compositions, include, but are not limited to: (a) VLA-4 antagonists such as those described in U.S. Pat. No. 5,510,332, WO95/15973, WO96/01644, WO96/06108, WO96/20216, WO96/22966, WO96/31206, WO96/40781, WO97/03094, WO97/02289, WO 98/42656, WO98/53814, WO98/53817, WO98/53818, WO98/54207, and WO98/58902; (b) steroids such as beclomethasone, methylprednisolone, betamethasone, prednisone, dexamethasone, and hydrocortisone; (c) immunosuppressants such as cyclosporin, tacrolimus, rapamycin, EDG receptor agonists including FTY-720, and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-UV); (i) other antagonists of the chemokine receptors, especially CCR-1, CCR-2, CCR-3, CXCR-3, CXCR-4 and CCR-5; (j) cholesterol lowering agents such as HMG-CoA reductase inhibitors (lovastatin, simvastatin and pravastatin, fluvastatin, atorvastatin, rosuvastatin, and other statins), sequestrants (cholestyramine and colestipol), cholesterol absorption inhibitors (ezetimibe), nicotinic acid, fenofibric acid derivatives (gemfibrozil, clofibrat, fenofibrate and benzafibrate), and probucol; (k) anti-diabetic agents such as insulin, sulfonylureas, biguanides (metformin), α-glucosidase inhibitors (acarbose) and glitazones (troglitazone and pioglitazone); (1) preparations of interferon beta (interferon beta-1α, interferon beta-1β); (m) preparations of glatiramer acetate; (n) preparations of CTLA4Ig; (o) preparations of hydroxychloroquine, (p) Copaxone® and (q) other compounds such as 5-aminosalicylic acid and prodrugs thereof, antimetabolites such as azathioprine, 6-mercaptopurine and methotrexate, leflunomide, teriflunomide, and cytotoxic and other cancer chemotherapeutic agents.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In treating, preventing, ameliorating, controlling or reducing the risk of conditions which require chemokine receptor modulation an appropriate dosage level will generally be about 0.0001 to 500 mg per kg patient body weight per day which can be administered in single or multiple doses. In certain embodiments the dosage level will be about 0.0005 to about 400 mg/kg per day; or from about 0.005 to about 300 mg/kg per day; or from about 0.01 to about 250 mg/kg per day, or from about 0.05 to about 100 mg/kg per day, or from about 0.5 to about 50 mg/kg per day. Within this range the dosage may be 0.0001 to 0.005, 0.005 to 0.05, 0.05 to 0.5, 0.5 to 5 or 5 to 50 mg/kg per day. For oral administration, the compositions may be provided in the form of tablets containing 0.01 to 1000 milligrams of the active ingredient, or 0.1 to 500, 1.0 to 400, or 2.0 to 300, or 3.0 to 200, particularly 0.01, 0.05, 0.1, 1, 4, 5, 10, 15, 20, 25, 30, 50, 75, 100, 125, 150, 175, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, or once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are commercially available, made by known procedures, or prepared as illustrated herein.

One of the principal routes used for preparation of compounds within the scope of the instant invention which bear a carbon substituted pyrazole framework 1-10 is depicted in Scheme 1. According to this route, appropriately substituted keto-esters 1-1 is reacted with hydrazines 1-2 with acetic acid at elevated temperatures to give pyrazole precursors 1-3. This compound is then converted to bromopyrazoles 1-4 using phosphorous tribromide. Bromopyrazoles 1-4 are further elaborated via a palladium catalyzed cross coupling with a suitable alkyne (1-5) to give carbon linked pyrazoles 1-6. These are then reduced using hydrogen and catalytic palladium to give compounds 1-7 in which the pendant alcohol is oxidized using pyridinium dichromate to yield pyrazole-acids 1-8. Pyrazole acids 1-8 are then coupled with appropriately substituted amines 1-9 using standard procedures to give the desired compounds 1-10. Amines 1-9 can exist as mixtures of stereoisomers, in which cases final products 1-10 are obtained as mixtures of stereoisomers. These can be separated by a variety of methods, including by HPLC using a column with a chiral stationary phase.

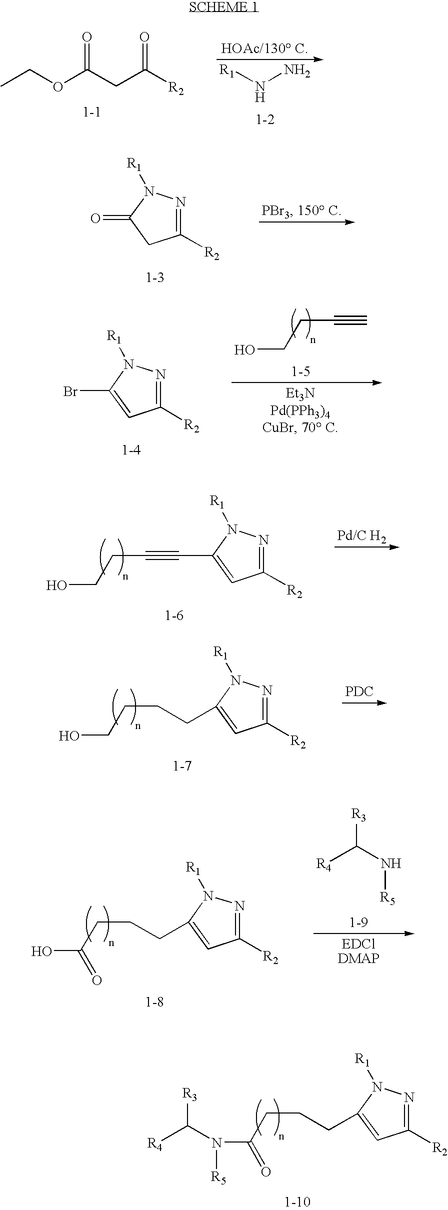

Another route used for preparation of compounds within the scope of the instant invention which bear an oxygen substituted pyrazole framework 2-4 is depicted in Scheme 2. According to this route, intermediates 1-3 are reacted with (3-bromopropoxy)-tert-butyldimethylsilane and an appropriate base to give oxygen substituted pyrazoles 2-1. These are then further reacted using standard methods for deprotection of the silyl group to give alcohols 2-2 and oxidation using an appropriate oxidant such as pyridinium dichromate to give acids 2-3. These are then coupled with appropriately substituted amines 1-9 using standard reagents such as EDCI or HATU to give the desired compounds 2-4. Amines 1-9 can exist as mixtures of stereoisomers, in which cases final products 2-4 are obtained as mixtures of stereoisomers. These can be separated by a variety of methods, including by HPLC using a column with a chiral stationary phase.

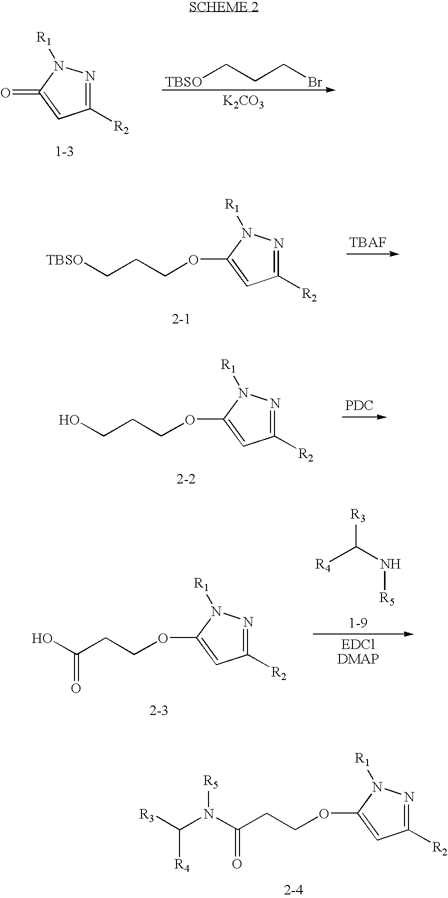

Another route used for preparation of compounds within the scope of the instant invention which bear a nitrogen substituted pyrazole framework 3-5 is depicted in Scheme 3. According to this route, cyanoketones 3-1 are reacted with hydrazines 1-2 to give nitrogen substituted pyrazoles 3-2. These are then reacted with ethyl 3-bromopropanoate and an appropriate base to give intermediates 3-3. The ester in intermediates 3-3 are then hydrolyzed to give acids 3-4 which are then coupled with appropriately substituted amines 1-9 using standard reagents such as EDCI or HATU to give the desired compounds 3-5. Amines 1-9 can exist as mixtures of stereoisomers, in which cases final products 3-5 are obtained as mixtures of stereoisomers. These can be separated by a variety of methods, including by HPLC using a column with a chiral stationary phase.

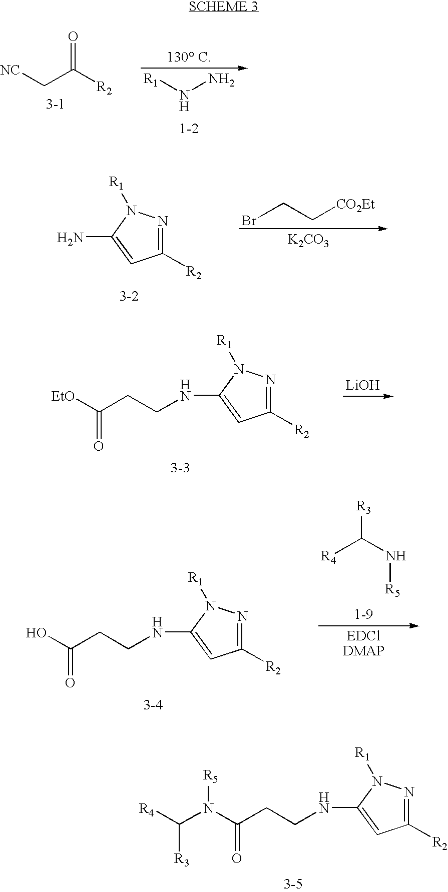

There are several more specialized ways to synthesize compounds of the formula I. These routes are elaborated in the experimental section. In some cases the order of carrying out the foregoing reaction schemes may be varied to facilitate the reaction or to avoid unwanted reaction products. The following examples are provided for the purpose of further illustration only and are not intended to be limitations on the disclosed invention.

Concentration of solutions was generally carried out on a rotary evaporator under reduced pressure. Flash chromatography was carried out on silica gel (230-400 mesh). Abbreviations: ethyl ether (ether), triethylamine (TEA), N,N-diisopropylethylamine (DIEA) saturated aqueous (sat'd), room temperature (rt), hour(s) (h), minute(s) (min).

The following are representative Procedures for the preparation of the compounds used in the following Examples or which can be substituted for the compounds used in the following Examples which may not be commercially available

### Intermediate 1

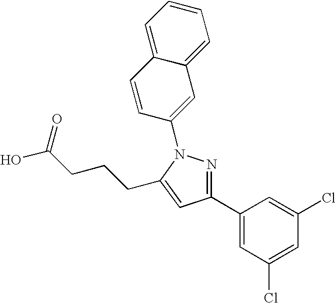

A mixture of ethyl 3-(3,5-dichlorophenyl)-3-oxopropanoate (500 mg, 1.9 mmol), 2-naphthylhydrazine hydrochloride (372 mg, 1.9 mmol) and glacial acetic acid (19 ml) was refluxed overnight and cooled to room temperature. Mixture was taken to pH 6 using 1.0N aqueous sodium hydroxide and then extracted with ethyl acetate, dried over sodium sulfate, filtered and concentrated. The crude material was purified by flash chromatography on silica gel (0-65% ethyl acetate/hexanes) to give 5-(3,5-dichlorophenyl)-2-(2-naphthyl)-2,4-dihydro-3H-pyrazol-3-one as a brown solid (365 mg, 54%). A mixture of 5-(3,5-dichlorophenyl)-2-(2-naphthyl)-2,4-dihydro-3H-pyrazol-3-one (360 mg, 1.0 mmol), phosphorous tribromide (3.0 ml, 32 mmol) and anhydrous acetonitrile (1.0 ml) was placed in a Smith microwavable vial and heated in the Smith microwave apparatus for two minutes at 100° C. to make mixture homogeneous, then heated to 150° C. until no starting material was observed by tlc. The reaction usually was completed in forty minutes. The mixture was carefully poured into ice and stirred at room temperature. Extracted mixture with ethyl acetate, dried over sodium sulfate, filtered and concentrated to give 5-bromo-3-(3,5-dichlorophenyl)-1-(2-naphthyl)-1H-pyrazole as a brown solid (425 mg, 100%). A mixture of 5-bromo-3-(3,5-dichlorophenyl)-1-(2-naphthyl)-1H-pyrazole (441 mg, 1.1 mmol), 3-butyn-1-ol (0.24 ml, 3.14 mmol), degassed triethylamine (10.2 ml), palladium tetrakistriphenylphosphine (194 mg, 0.17 mmol) and copper (I) bromide dimethyl sulfide (65 mg, 0.32 mmol) was heated to 70° C. for a few hours until no starting material was observed by tlc. Mixture was cooled, concentrated, washed with brine and extracted with ethyl acetate. Organic extracts were combined, dried over sodium sulfate, filtered and concentrated to give a crude material which was further purified via flash chromatography on silica gel (0-75% ethyl acetate/hexanes). This afforded 4-[3-(3,5-dichlorophenyl)-1-H pyrazol-5-yl]but-3-yn-1-ol as a tan solid (318 mg, 74%). 4-[3-(3,5-Dichlorophenyl)-1-H pyrazol-5-yl]but-3-yn-1-ol (318 mg, 0.8 mmol), 5% platinum on carbon (200 mg) and ethyl acetate (20 ml) was stirred under hydrogen atmosphere at one atmosphere of pressure overnight. The mixture was filtered through a celite pad, washing with ethyl acetate and the filtrate concentrated to give 4-[3-(3,5-dichlorophenyl)-1-(2-naphthyl)-1H-pyrazol-5-yl]butan-1-ol as a white solid (255 mg, 80%). A mixture of 4-[3-(3,5-dichlorophenyl)-1-(2-naphthyl)-1H-pyrazol-5-yl]butan-1-ol (255 mg, 0.62 mmol), pyridinium dichromate (816 mg, 2.17 mmol) and dimethylformamide (3.1 ml) was stirred at room temperature overnight. Purification of the reaction mixture via flash chromatography on silica gel (0-40% ethyl acetate/hexanes) afforded 4-[3-(3,5-dichlorophenyl)-1-(2-naphthyl)-1H pyrazol-5-yl]butanoic acid as a white solid (159 mg, 60%). MS (ESI) 426 (M⁺).

### EXAMPLE 1

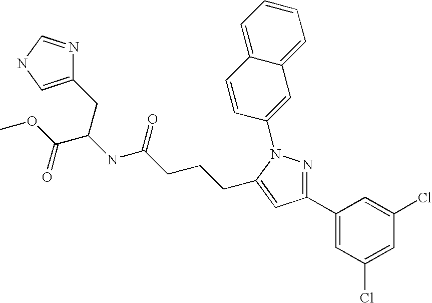

A mixture of 4-[3-(3,5-dichlorophenyl)-1-(2-naphthyl)-1H pyrazol-5-yl]butanoic acid (29 mg, 0.07 mmol), methyl 1-tritylhistidinate hydrochloride (30 mg, 0.07 mmol), dimethylaminopyridine (43 mg, 0.35 mmol), EDCI hydrochloride (27 mg, 0.14 mmol) in dichloromethane (3.5 ml) was stirred at 0° C. and then warmed to room temperature overnight. Mixture was washed with water, extracted with ethyl acetate, dried over sodium sulfate, filtered and concentrated in vacuo. The crude material was dissolved in dichloromethane (2.0 ml) and treated with trifluoroacetic acid (0.2 ml, 2.5 mmol). The mixture was stirred at room temperature until no starting material was observed by LC/MS and then concentrated in vacuo. The crude oil was purified via reverse phase prep HPLC to give methyl N-{4-[3-(3,5-dichlorophenyl)-1-(2-naphthyl)-1H-pyrazol-5-yl]butanoyl}histidinate as a oil (19 mg, 34%). MS (ESI) 576 (M⁺).

### EXAMPLE 2

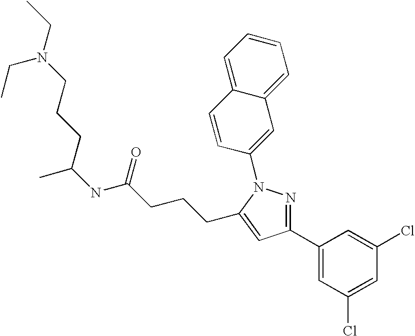

4-[3-(3,5-Dichlorophenyl)-1-(2-naphthyl)-1H pyrazol-5-yl]butanoic acid (25 mg, 0.06 mmol), dimethylformamide (1.0 ml), 2-amino-5-diethylaminopentane (0.01 ml, 0.06 mmol), dimethylamino-pyridine (28 mg, 0.23 mmol) and EDCI hydrochloride (22 mg, 0.12 mmol) was stirred at room temperature overnight under nitrogen. The mixture was washed with brine and extracted with ethyl acetate. Organic extracts were combined and concentrated to give a crude oil which was purified by reverse phase prep HPLC to give 4-[3-(3,5-dichlorophenyl)-1-(2-naphthyl)-1H-pyrazol-5-yl]-N-[4-diethylamino)-1-methylbutyl]butanamide as an oil (24 mg, 52%). MS (ESI) 567 (M+).

### EXAMPLE 3

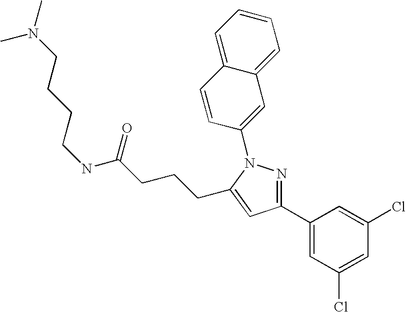

A similar procedure as outlined in example 2 was followed with 4-(dimethyl amine)butylamine to give 4-[3-(3,5-dichlorophenyl)-1-(2-naphthyl)-1H-pyrazole-5-yl]-N-[4-(dimethylamino)butyl]butanamide as a clear oil. MS (ESI) 524 (M⁺+H), 523 (M⁺).

### EXAMPLE 4

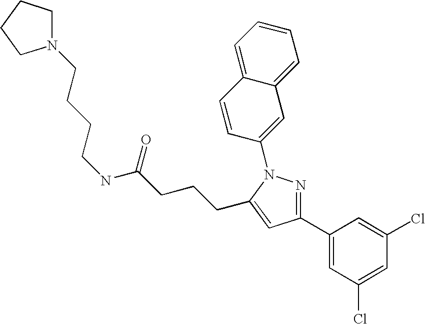

A similar procedure as outlined in example 2 was followed with 1-(4-aminobutyl)pyrrolidine to give 4-[3-(3,5-dichlorophenyl)-1-(2-naphthyl)-1H-pyrazol-5-yl]-N-(4-pyrrolidin-1-ylbutyl)butanamide as an oil. MS (ESI) 550 (M⁺+H), 549 (M⁺).

### EXAMPLE 5

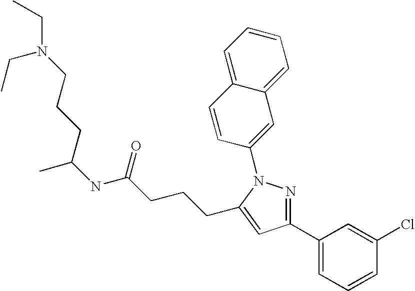

A similar procedure as outlined for intermediate 1 and example 2 was followed with ethyl (3-chlorobenzoyl)acetate to give 4-[3-(3-chlorophenyl)-1-(2-naphthyl)-1H-pyrazol-5-yl]-N-[4-(diethylamino)-1-methylbutyl]butanamide as tan solid. MS (ESI) 534, 533 (M⁺+2H).

### EXAMPLE 6

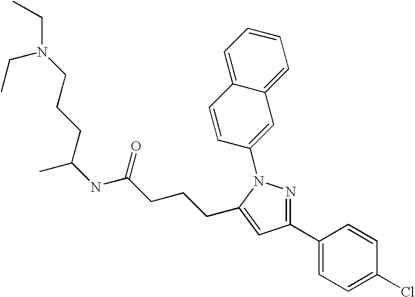

A similar procedure as outlined for intermediate 1 and example 2 was followed with methyl (4-chlorobenzoyl)acetate to give 4-[3-(4-chlorophenyl)-1-(2-napthyl)-1H-pyrazol-5-yl]-N-[4-(diethylamino)-1-methylbutyl]butanamide as a colorless oil. MS (ESI) 533 (M⁺+2H), 531 (M⁺).

### EXAMPLE 7

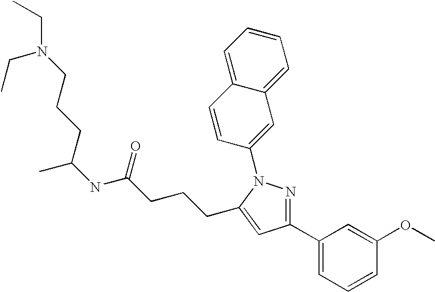

A similar procedure as outlined for intermediate 1 and example 2 was followed with ethyl (3-methoxybenzoyl)acetate to give N-[4-diethylamino)-1-methylbutyl]-4-[3-(3-methoxyphenyl)-1-(2-naphthyl)-1H-pyrazol-5-yl]butanamide as an oil. MS (ESI) 528 (M⁺).

### EXAMPLE 8

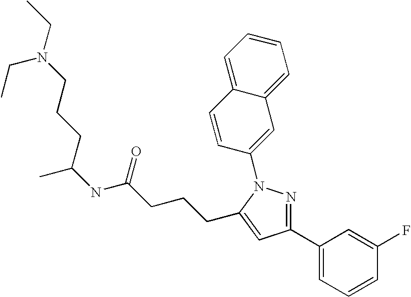

A similar procedure was outlined for intermediate 1 and example 2 was followed with ethyl (3-fluorobenzoyl)acetate to give N-[4-(diethylamino)-1-methylbutyl]-4-[3-(3-fluorophenyl)-1-(2-naphthyl)-1H-pyrazol-5-yl]butanamide as a colorless oil. MS (ESI) 516 (M⁺+2H), 515 (M⁺).

### EXAMPLE 9

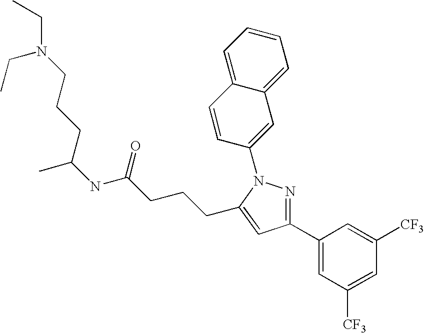

A similar procedure was outlined for intermediate 1 and example 2 was followed with ethyl [3,5-bis(trifluoromethyl)benzoyl]acetate to give 4-[3-[3,5-bis(trifluoromethyl)phenyl]-1-(2-naphthyl)-1H-pyrazol-5-yl]-N-[4-(diethylamino)-1-methylbutyl]butanamide as an oil. MS (ESI) 634 (M⁺+1).

### EXAMPLE 10

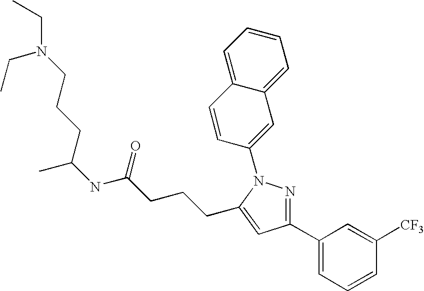

A similar procedure was outlined for intermediate 1 and example 2 was followed with methyl-3-trifluoromethyl benzoyl acetate to give N-[4-(diethylamino)-1-methylbutyl]-4-{1-(2-naphthyl)-3-[3-(trifluoromethyl)phenyl]-1H-pyrazol-5-yl}butanamide as an oil.

MS (ESI) 566 (M⁺+1), 565 (M⁺).

### EXAMPLES 11-13

Following the procedure described in Example 2, a series of analogous target compounds were synthesized. Their structure and MS-characteristics are summarized in the following Table.

TABLE 1

| EXAMPLE | Structure | MS (ESI) | name |
|---|---|---|---|
| 11 | 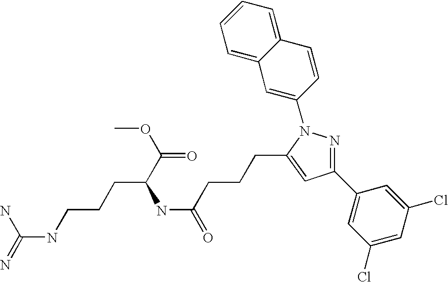 | MS 595.44(M⁺ + H). | Methyl N²-{4-[3-(3,5-dichlorophenyl)-1-(2-naphthyl)-1H-pyrazol-5-yl]butanoyl}-L-argininate |
| 12 | 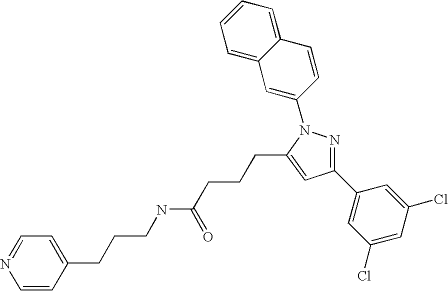 | MS 543.57(M⁺ + H). | 4-[3-(3,5-dichlorophenyl)-1-(2-naphthyl)-1H-pyrazol-5-yl]-N-(3-pyridin-4-ylpropyl)butanamide |
| 13 | 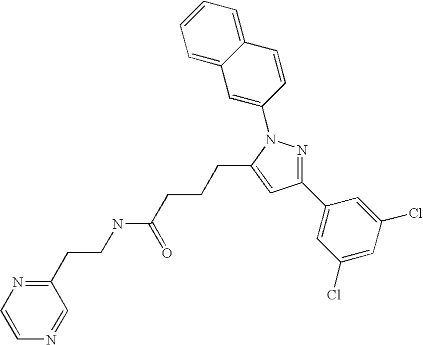 | MS 530.31(M⁺ + H). | 4-[3-(3,5-dichlorophenyl)-1-(2-naphthyl)-1H-pyrazol-5-yl]-N-(2-pyrazin-2-ylethyl)butanamide |

### Intermediate 2

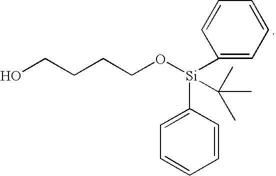

To a 0° C. solution of butane-1,4-diol (9.00 g, 100.00 mmol) and 1H-imidazole (6.8 g, 100.00 mmol), in 150 ml of N,N-dimethylformamide was added tert-butyl(chloro)diphenylsilane over 1 hour. The result solution was stirring at room temperature for 12 hour. The reaction mixture was quenched with H₂O (100 mL), then extracted with EtOAc (3×100 mL) and the combined organic extracts washed with brine. The organic phase was dried over Na₂SO₄ and concentrated in vacuo. The crude purified by liquid chromatography on silica gel using an ISCO single channel system (Hexane/EtOAc:9/1 to 1/1) to give 4-{[tert-butyl(diphenyl)silyl]oxy}butan-1-ol. ¹HNMR (CD₃Cl₃, 500 MHz) δ 7.69-7.66 (m, 4H), 7.43-7.35 (m, 6H), 3.71-3.68 (t, 2H), 3.65-3.63 (t, 2H), 2.33 (d, 1H), 1.69-1.62 (m, 4H), 1.05 (s, 9H).

### Intermediate 3

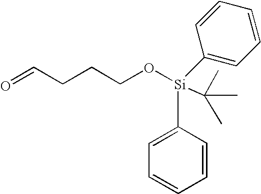

To a 0° C. solution of 4-{[tert-butyl(diphenyl)silyl]oxy}butan-1-ol (13.12 g, 40.00 mmol), 4-Methylmorpholine (5.14 g, 44.00 mmol) and 5 g 4 Å molecular sieve in 100 ml of mixed solvent of CH₂Cl₂ and CH₃CN (V:V=1:3) was added Tetrapropylammonium perruthenate (100 mg). The result solution was stirring at room temperature for 12 hour. The reaction mixture was concentrated in vacuo and directly load on silica gel, purified on ISCO single channel system (Hexane/EtOAc 10/0:9/1) to give 4-{[tert-butyl(diphenyl)silyl]oxy}butanal as product. ¹H NMR (CD₃Cl₃, 500 MHz) δ 9.78 (s, 1H), 7.65-7.63 (m, 4H), 7.42-7.25 (m, 6H), 3.70-3.68 (t, 2H), 2.56-2.53 (m, 2H), 1.91-1.86 (m, 2H), 1.04 (s, 9H).

### Intermediate 4

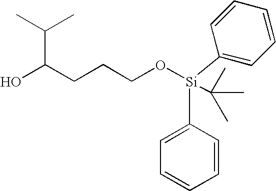

To a 0° C. solution of 4-{[tert-butyl(diphenyl)silyl]oxy}butanal (3.26 g, 10 mmol) in 30 ml THF was added Isopropyl magnesium bromide (12 ml, 12 mmol). The result solution was stirring for 2 hours and allowed warm to room temperature. The reaction mixture was quenched with H₂O (30 mL), then extracted with EtOAc (3×30 mL) and the combined organic extracts washed with brine. The organic phase was dried over Na₂SO₄ and concentrated in vacuo. The crude purified by liquid chromatography on silica gel using an ISCO single channel system (Hexane/EtOAc:10/0 to 1/9) to give 6-{[tert-butyl(diphenyl)silyl]oxy}-2-methylhexan-3-ol as product. ¹H NMR (CD₃Cl₃, 500 MHz) δ 7.68-7.63 (m, 4H), 7.43-7.34 (m, 6H), 3.71-3.69 (t, 2H), 3.39-3.35 (m, 1H), 1.92 (S, Br, 1H), 1.66-1.63 (m, 4H), 1.40-1.50 (m, 1H), 1.05 (s, 9H), 0.92-0.90 (d, 6H).

### Intermediate 5

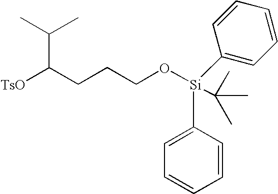

To the 0° C. solution of 6-{[tert-butyl(diphenyl)silyl]oxy}-2-methylhexan-3-ol (1.85 g, 5 mmol) in 5 ml of pyridine was added 4-methylbenzenesulfonyl chloride. The result solution was stirring at room temperature for 12 hour. The reaction mixture was quenched with H₂O (100 mL), then extracted with EtOAc (3×1100 mL) and the combined organic extracts washed with brine. The organic phase was dried over Na₂SO₄ and concentrated in vacuo. The crude purified by liquid chromatography on silica gel using an ISCO single channel system (Hexane/EtOAc:10/0 to 1/1) to give 4-{[tert-butyl(diphenyl)silyl]oxy}-1-isopropylbutyl 4-methylbenzenesulfonate as product. ¹H NMR (CD₃Cl₃, 500 MHz) δ 7.77-7.75 (d, 2H), 7.65-7.60 (m, 4H), 7.43-7.34 (m, 6H), 7.27-7.25 (d, 1H), 4.49-4.45 (m, 1H), 3.57-3.54 (t, 2H), 2.39 (s, 3H), 1.94-1.92 (m, 1H), 1.67-1.51 (m, 2H), 1.55-1.35 (m, 2H), 1.03 (s, 9H), 0.88-0.85 (d, 6H).

### Intermediate 6

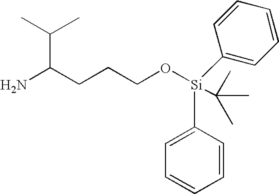

The solution of 4-{[tert-butyl(diphenyl)silyl]oxy}-1-isopropylbutyl 4-methylbenzenesulfonate (1.075 g, 2.05 mmol), Sodium Azide (147 mg, 2.25 mmol) in 5 ml N,N-dimethylformamide was stirred at 40° C. for 12 hour. The reaction mixture was quenched with H₂O (30 mL), then extracted with EtOAc (3×30 mL) and the combined organic extracts washed with brine. The organic phase was dried over Na₂SO₄ and concentrated in vacuo. The crude purified by liquid chromatography on silica gel using an ISCO single channel system (Hexane/EtOAc:10/0 to 1/1) to give [(4-azido-5-methylhexyl)oxy](tert-butyl)diphenylsilane.

The solution of [(4-azido-5-methylhexyl)oxy](tert-butyl)diphenylsilane (0.395 g, 1 mmol), 20 mg of palladium on carbon (10% W/W) in 20 ml mixed solvent of methanol ethyl acetate (V:V=1:1) was treated with and hydrogen gas for overnight. The reaction mixture was filtrated over celite, concentrated to give (4-{[tert-butyl(diphenyl)silyl]oxy}-1-isopropylbutyl)amine as product. ¹H NMR (CD₃Cl₃, 500 MHz) δ 7.68-7.66 (m, 4H), 7.43-7.35 (m, 6H), 3.70-3.64 (m, 2H), 2.50-2.47 (m, 1H), 1.71-1.67 (m, 1H), 1.58-1.53 (m, 3H), 1.27-1.24 (m, 1H), 1.05 (s, 9H), 0.89-0.84 (m, 6H). MS (ESI) 370.72 (M⁺+H).

### Intermediate 7

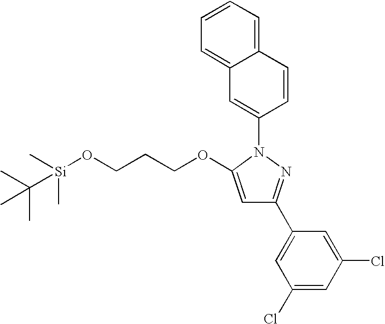

The solution of 5-(3,5-dichlorophenyl)-2-(2-naphthyl)-2,4-dihydro-3H-pyrazol-3-one (200 mg, 0.565 mg), (3-bromopropoxy)-tert-butyldimethylsilane (285 mg, 1.13 mmol), potassium carbonate (156 mg, 1.13 mmol) in 3 ml N,N-dimethylformamide was stirred at 60° C. for 3 hours. The reaction mixture was directly load on silica and purified by liquid chromatography on silica gel using an ISCO single channel system (Hexane/EtOAc:10/0 to 1/1) to give 5-(3-{[tert-butyl(dimethyl)silyl]oxy}propoxy)-3-(3,5-dichlorophenyl)-1-(2-naphthyl)-1H-pyrazole as product. ¹H NMR (CD₃Cl₃, 500 MHz) δ 8.19 (s, 1H), 7.95-7.93 (dd, 1H), 7.89-7.83 (m, 3H), 7.745-7.742 (m, 2H), 7.50-7.46 (m, 2H), 7.28-7.23 (m, 1H), 6.01 (s, 1H), 4.32-4.29 (t, 2H), 3.78-3.76 (t, 2H), 2.04-2.02 (m, 2H), 0.86 (s, 9H), 0.02 (s, 6H).

### Intermediate 8

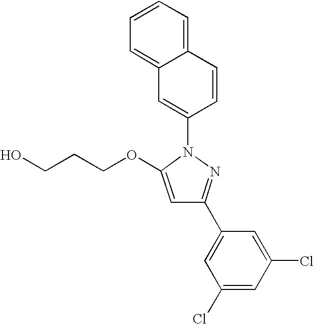

The solution of 5-(3-{[tert-butyl(dimethyl)silyl]oxy}propoxy)-3-(3,5-dichlorophenyl)-1-(2-naphthyl)-1H-pyrazole (0.206 g, 0.500 mmol) in 5 ml THF was treated with 0.565 ml of Tetrabutylammonium fluoride (1M in THF) for 5 minute. The reaction mixture was quenched with H₂O (20 mL), then extracted with EtOAc (3×20 mL) and the combined organic extracts washed with brine. The organic phase was dried over Na₂SO₄ and concentrated in vacuo. The crude purified by liquid chromatography on silica gel using an ISCO single channel system (Hexane/EtOAc:10/0 to 1/9) to give 3-{[3-(3,5-dichlorophenyl)-1-(2-naphthyl)-1H-pyrazol-5-yl]oxy}propan-1-ol as product. MS (ESI) 413.0 (M+H). ¹HNMR (CD₃Cl₃, 500 MHz) δ 8.174-8.171 (d, 1H), 7.94-7.91 (dd, 1H), 7.89-7.83 (m, 3H), 7.75-7.74 (m, 2H), 7.51-7.46 (m, 2H), 7.30-7.29 (m, 1H), 5.98 (s, 1H), 4.30-4.27 (t, 2H), 3.81-3.78 (t, 2H), 2.08-2.03 (m, 2H).

### Intermediate 9

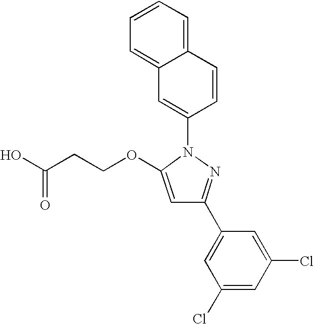

The solution of 3-{[3-(3,5-dichlorophenyl)-1-(2-naphthyl)-1H-pyrazol-5-yl]oxy}propan-1-ol (152 mg, 0.369 mg), pyridinium dichromate (485 mg, 1.29 mmol) in 4 ml N,N-dimethylformamide was stirred at room temperature for 12 hour. The reaction mixture was quenched with H₂O (20 mL), then extracted with EtOAc (3×20 mL) and the combined organic extracts washed with brine. The organic phase was dried over Na₂SO₄ and concentrated in vacuo. The crude purified by liquid chromatography on silica gel using an ISCO single channel system (Hexane/EtOAc:9/1 to 1/9) to give 3-{[3-(3,5-dichlorophenyl)-1-(2-naphthyl)-1H-pyrazol-5-yl]oxy}propanoic acid as product. ¹H NMR (CD₃Cl₃, 500 MHz) δ 8.22 (s, 1H), 7.98-7.96 (m, 2H), 7.93-7.88 (m, 2H), 7.79-7.78 (m, 2H), 7.56-7.50 (m, 2H), 7.34-7.33 (m, 1H), 6.06 (s, 1H), 4.32-4.30 (t, 2H), 2.62-2.59 (t, 2H), 2.26-2.20 (m, 2H). MS (ESI) 441.18 (M⁺+Na).

### Intermediate 10

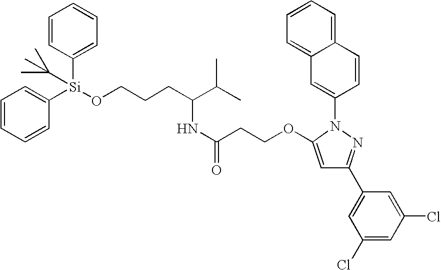

The solution of (4-{[tert-butyl(diphenyl)silyl]oxy}-1-isopropylbutyl)amine (0.127 g, 0.3 mmol), 3-{[3-(3,5-dichlorophenyl)-1-(2-naphthyl)-1H-pyrazol-5-yl]oxy}propanoic acid (0.111 g, 0.3 mmol), 1-(3-Dimethylaminopropyl)-3-ethylcarbodiimide hydrochloride (0.0573 g, 0.3 mmol), 4-Dimethylaminopyridine (0.0369 g, 0.3 mmol) in 2 ml N,N-dimethylformamide was stirred at 40° C. for 12 hour. The reaction mixture was quenched with H₂O (20 mL), then extracted with EtOAc (3×20 mL) and the combined organic extracts washed with brine. The organic phase was dried over Na₂SO₄ and concentrated in vacuo. The crude purified by liquid chromatography on silica gel using an ISCO single channel system (Hexane/EtOAc:10/0 to 1/9) to give N-(4-{[tert-butyl(diphenyl)silyl]oxy}-1-isopropylbutyl)-3-{[3-(3,5-dichlorophenyl)-1-(2-naphthyl)-1H-pyrazol-5-yl]oxy}propanamide as product. ¹HNMR (CD₃Cl₃, 500 MHz) δ 8.139-8.136 (d, 1H), 7.87-7.82 (m, 3H), 7.79-7.78 (m, 1H), 7.76-7.75 (d, 2H), 7.62-7.60 (m, 4H), 7.46-7.43 (m, 2H), 7.40-7.38 (m, 2H), 7.37-7.33 (m, 4H), 7.314-7.307 (m, 1H), 6.05 (s, 1H), 5.26-5.24 (d, 1H), 4.54-4.47 (m, 2H), 3.78-3.75 (m, 1H), 3.56-3.47 (m, 2H), 2.67-2.65 (t, 2H), 1.56-1.53 (m, 2H), 1.42-1.39 (m, 2H), 1.15-1.05 (m, 1H), 1.01 (s, 9H), 0.74-0.73 (d, 3H), 0.70-0.68 (d, 3H).

### Intermediate 11

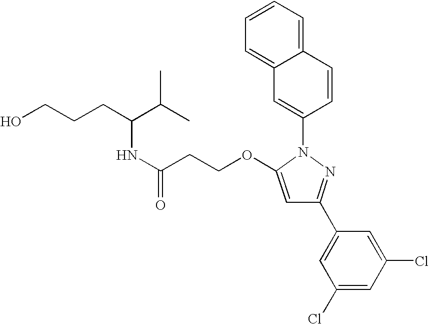

The solution of N-(4-{[tert-butyl(diphenyl)silyl]oxy}-1-isopropylbutyl)-3-{[3-(3,5-dichlorophenyl)-1-(2-naphthyl)-1H-pyrazol-5-yl]oxy}propanamide (87 mg, 0.112 mmol) in THF was added 2 ml Tetrabutylammonium fluoride (1M in THF). After 10 minute, the reaction mixture was quenched with H₂O (20 mL), then extracted with EtOAc (3×20 mL) and the combined organic extracts washed with brine. The organic phase was dried over Na₂SO₄ and concentrated in vacuo. The crude purified by liquid chromatography on silica gel using an ISCO single channel system (Hexane/EtOAc:9/1 to 1/9) to give 3-{[3-(3,5-dichlorophenyl)-1-(2-naphthyl)-1H-pyrazol-5-yl]oxy}-N-(4-hydroxy-1-isopropylbutyl)propanamide as product. ¹H NMR (CD₃Cl₃, 500 MHz) δ 8.17 (s, 1H), 7.91-7.85 (m, 4H), 7.766-7.762 (m, 2H), 7.54-7.46 (m, 2H), 7.32-7.31 (m, 1H), 6.09 (s, 1H), 5.37-5.35 (d, 1H), 4.57-4.53 (m, 2H), 3.86-3.77 (m, 1H), 3.48-3.46 (m, 2H), 2.75-2.72 (t, 2H), 1.56-1.37 (m, 4H), 1.15-1.05 (m, 1H), 0.764-0.750 (d, 3H), 0.717-0.703 (d, 3H). MS (ESI) 540.56 (M⁺+H).

### EXAMPLE 14

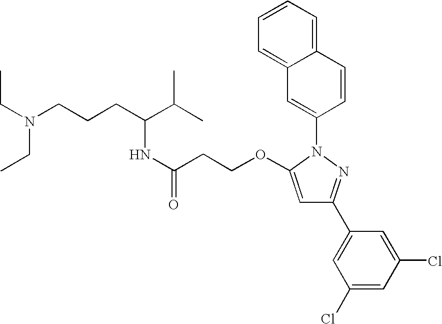

To the solution of 3-{[3-(3,5-dichlorophenyl)-1-(2-naphthyl)-1H-pyrazol-5-yl]oxy}-N-(4-hydroxy-1-isopropylbutyl)propanamide (48 mg, 0.089 mmol) in 3 ml Dichloromethane was added Dess-Martin periodinane (41.5 mg, 0.098 mmol). After the reaction was stirred at room temperature for 4 hr, LC-MS indicate starting material was consumed, removing solvent to give crude product 3-{[3-(3,5-dichlorophenyl)-1-(2-naphthyl)-1H-pyrazol-5-yl]oxy}-N-(1-isopropyl-4-oxobutyl)propanamide which was used for next step without further purification. To the solution of crude product 3-{[3-(3,5-dichlorophenyl)-1-(2-naphthyl)-1H-pyrazol-5-yl]oxy}-N-(1-isopropyl-4-oxobutyl)propanamide in mixed solvent of 1 ml THF, 0.2 ml of Acetic acid was added diethyl amine (0.707 g, 9.6 mmol), Sodium cyanideborohydride (20.0 mg, 0.318 mmol). The result solution was stirring at room temperature for 4 hour. The reaction mixture was quenched with H₂O (20 mL), then extracted with EtOAc (3×20 mL) and the combined organic extracts washed with brine. The organic phase was dried over Na₂SO₄ and concentrated in vacuo. The crude purified by reverse phase HPLC to give 3-{[3-(3,5-dichlorophenyl)-1-(2-naphthyl)-1H-pyrazol-5-yl]oxy}-N-[4-(diethylamino)-1-isopropylbutyl]propanamide as product. ¹H NMR (MeOD, 500 MHz) δ 8.25 (s, 1H), 7.98-7.95 (m, 3H), 7.92-7.90 (m, 1H), 7.87-7.83 (m, 2H), 7.57-7.51 (m, 2H), 7.41-7.40 (m, 1H), 6.45 (s, 1H), 4.61-4.58 (m, 2H), 3.64-3.62 (m, 1H), 2.88-2.74 (m, 8H), 1.59-1.56 (m, 1H), 1.49-1.41 (m, 1H), 1.40-1.20 (m, 4H), 1.10-1.16 (t, 6H), 0.786-0.752 (m, 6H). MS (ESI) 595.68 (M⁺+H).

### EXAMPLES 15-22

Following the procedure described in Example 2 using intermediate 9, a series of analogous target compounds were synthesized. Their structure and MS-characteristics are summarized in the following Table.

TABLE 2

| EXAMPLE | Structure | MS (ESI) | Name |
|---|---|---|---|
| 15 | 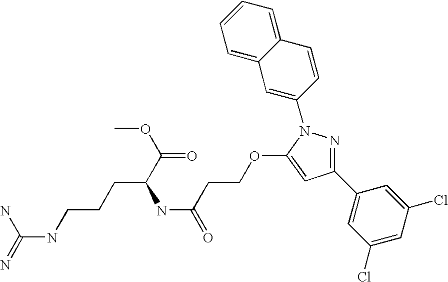 | MS 597.42(M⁺ + H) | Methyl N²-(3-{[3-(3,5-dichlorophenyl)-1-(2-naphthyl)-1H-pyrazol-5-yl]oxy}propanoyl)-L-argininate |
| 16 | 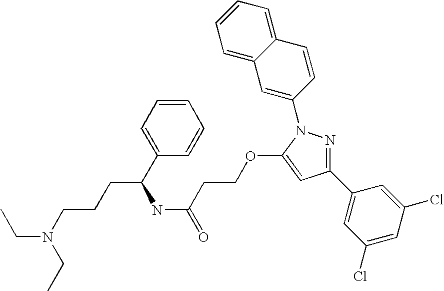 | MS 629.16(M⁺ + H). | 3-{[3-(3,5-dichlorophenyl)-1-(2-naphthyl)-1H-pyrazol-5-yl]oxy}-N-[(1S)-4-(diethylamino)-1-phenylbutyl]propanamide |
| 17 | 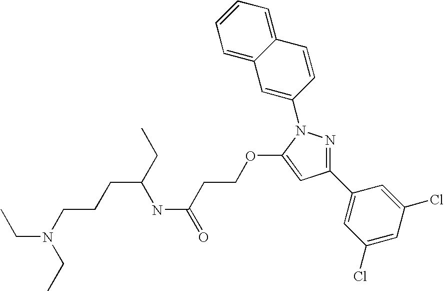 | MS 581.58(M⁺ + H). | 3-{[3-(3,5-dichlorophenyl)-1-(2-naphthyl)-1H-pyrazol-5-yl]oxy}-N-[4-(diethylamino)-1-ethylbutyl]propanamide |
| 18 | 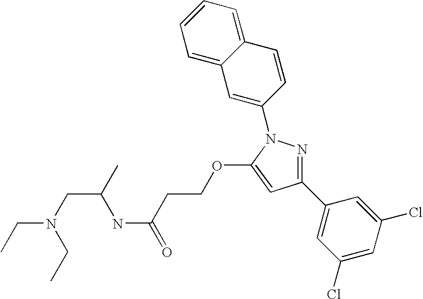 | MS 539.69(M⁺ + H). | 3-{[3-(3,5-dichlorophenyl)-1-(2-naphthyl)-1H-pyrazol-5-yl]oxy}-N-[2-(diethylamino)-1-methylethyl]propanamide |
| 19 | 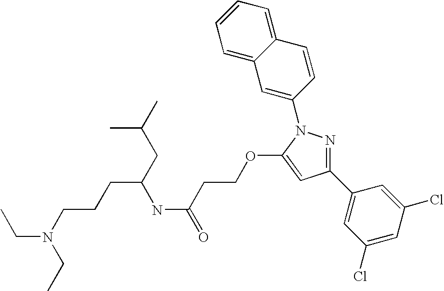 | MS 609.20(M⁺ + H). | 3-{[3-(3,5-dichlorophenyl)-1-(2-naphthyl)-1H-pyrazol-5-yl]oxy}-N-{1-[3-(diethylamino)propyl]-3-methylbutyl}propanamide |
| 20 | 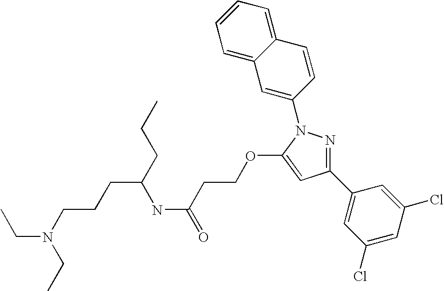 | MS 595.10(M⁺ + H). | 3-{[3-(3,5-dichlorophenyl)-1-(2-naphthyl)-1H-pyrazol-5-yl]oxy}-N-[4-(diethylamino)-1-propylbutyl]propanamide |
| 21 | 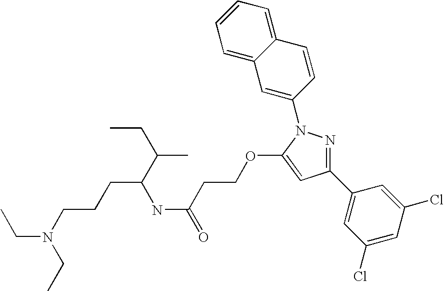 | MS 609.20(M⁺ + H). | 3-{[3-(3,5-dichlorophenyl)-1-(2-naphthyl)-1H-pyrazol-5-yl]oxy}-N-{1-[3-(diethylamino)propyl]-2-methylbutyl}propanamide |
| 22 | 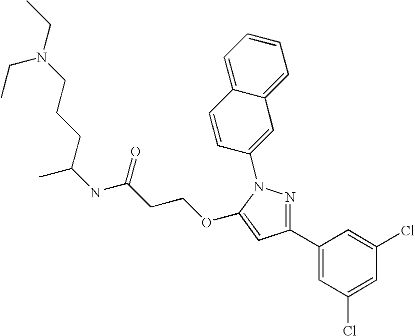 | MS 569(M⁺ + 2 H),567 (M⁺). | 3-{[3-(3,5-dichlorophenyl)-1-(2-naphthyl)-1H-pyrazol-5-yl]oxy}-N-[4-(diethylamino)-1-methylbutyl]propanamide |

### Intermediate 12

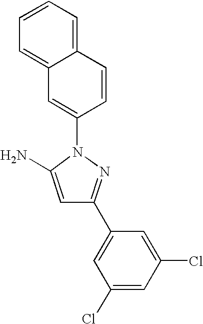

The mixture of 2-naphthylhydrazine hydrochloride (1.81 g, 9.33 mmol), 3-(3,5-dichlorophenyl)-3-oxopropanenitrile (2.44 g, 11.45 mmol) in 50 ml toluene was heated to 130° C. for 12 hours. The reaction mixture was quenched with aqueous sodium bicarbonate (100 mL), extracted with EtOAc (3×100 mL) and the combined organic extracts washed with brine. The organic phase was dried over Na₂SO₄ and concentrated in vacuo. The crude purified by liquid chromatography on silica gel using an ISCO single channel system (Hexane/EtOAc:10/0 to 1/9) to give 3-(3,5-dichlorophenyl)-1-(2-naphthyl)-1H-pyrazol-5-amine as product. ¹HNMR (CD₃Cl₃, 500 MHz) δ 8.055-8.051 (d, 1H), 8.00-7.98 (d, 1H), 7.90-7.88 (m, 2H), 7.78-7.60 (dd, 1H), 7.726-7.722 (m, 2H), 7.57-7.53 (m, 2H), 7.29-7.28 (t, 1H), 5.96 (s, 1H), 3.95 (s, 2H). MS (ESI) 353.97 (M⁺+H).

### Intermediate 13

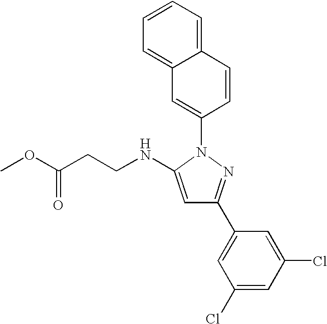

The mixture of 3-(3,5-dichlorophenyl)-1-(2-naphthyl)-1H-pyrazol-5-amine (0.353 g, 1.0 mmol), methyl 3-bromopropanoate (3.30 g, 10 mmol), potassium carbonate (0.276 g, 2.0 mmol), sodium iodide (44.9 mg, 0.3 mmol) in 5 ml N,N-dimethylformamide was heated to 200° C. under microwave irradiation. The reaction mixture was quenched with H₂O (20 mL), then extracted with EtOAc (3×20 mL) and the combined organic extracts washed with brine. The organic phase was dried over Na₂SO₄ and concentrated in vacuo. The crude purified by liquid chromatography on silica gel using an ISCO single channel system (Hexane/EtOAc:10/0 to 1/9) to give methyl N-[3-(3,5-dichlorophenyl)-1-(2-naphthyl)-1H-pyrazol-5-yl]-β-alaninate as product. ¹H NMR (CD₃Cl₃, 500 MHz) δ 8.009-8.005 (m, 1H), 7.98-7.96 (d, 1H), 7.89-7.88 (m, 2H), 7.743-7.440 (m, 2H), 7.72-7.70 (m, 1H), 7.57-7.52 (m, 2H), 7.29-7.28 (m, 1H), 5.88 (s, 1H), 3.66 (s, 3H), 3.48-3.46 (t, 2H), 2.69-2.67 (t, 2H). MS (ESI) 440.17 (M⁺+H).

### Intermediate 14

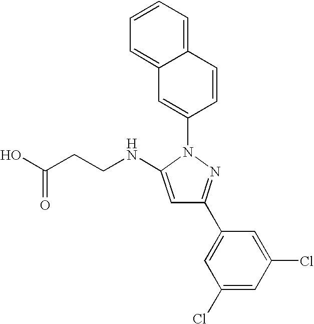

The solution of ethyl N-[3-(3,5-dichlorophenyl)-1-(2-naphthyl)-1H-pyrazol-5-yl]-β-alaninate (0.453 g, 1.0 mmol), lithium hydroxide (5 ml 1M solution, 5.0 mmol) in mixed solvent of 10 ml methanol and THF (V:V=9:1) was heated for 2 hours at 70° C. The reaction mixture was quenched with H₂O (20 mL), then extracted with EtOAc (3×20 mL) and the combined organic extracts washed with brine. The organic phase was dried over Na₂SO₄ and concentrated in vacuo. The crude purified by liquid chromatography on silica gel using an ISCO single channel system (Hexane/EtOAc:9/1 to 1/9) to give N-[3-(3,5-dichlorophenyl)-1-(2-naphthyl)-1H-pyrazol-5-yl]-β-alanine. ¹H NMR (CD₃Cl₃, 500 MHz) δ 7.967-7.964 (m, 1H), 7.94-7.92 (d, 1H), 7.87-7.82 (m, 2H), 7.736-7.732 (m, 2H), 7.69-7.67 (dd, 1H), 7.52-7.50 (m, 2H), 7.29-7.28 (m, 1H), 5.87 (s, 1H), 3.66 (s, 3H), 3.47-3.44 (t, 2H), 2.70-2.68 (t, 2H). MS (ESI) 428.05 (M⁺+H).

### EXAMPLE 23

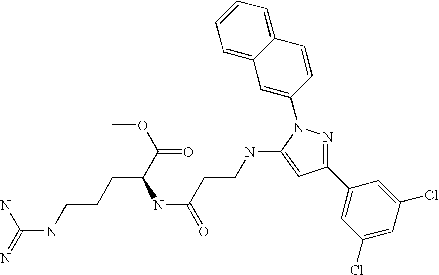

The mixture of N-[3-(3,5-dichlorophenyl)-1-(2-naphthyl)-1H-pyrazol-5-yl]-β-alanine (42.5 mg, 0.10 mmol), methyl L-argininate dihydrochloride, (52.2 mg, 0.2 mmol), 1-(3-Dimethylaminopropyl)-3-ethylcarbodiimide hydrochloride (38.3 mg, 0.2 mmol), 4-Dimethylaminopyridine (73.3 mg, 0.6 mmol) in 2 ml N,N-dimethylformamide was stirred at 40° C. for 12 hour. The reaction mixture was quenched with H₂O (20 mL), then extracted with EtOAc (3×20 mL) and the combined organic extracts washed with brine. The organic phase was dried over Na₂SO₄ and concentrated in vacuo. The crude was purified by reverse phase HPLC to give Methyl N-[3-(3,5-dichlorophenyl)-1-(2-naphtlyl)-1H-pyrazol-5-yl]-p-alanyl-L-argininate as product. ¹H NMR (MD₃OD), 500 MHz) δ 8.082-8.079 (d, 1H), 8.04-8.02 (d, 1H), 7.97-7.93 (m, 2H), 7.797-7.794 (m, 2H), 7.73-7.70 (m, 1H), 7.57-7.54 (m, 2H), 7.386-7.379 (t, 1H), 4.42-4.40 (m, 1H), 3.60 (s, 3H), 3.51-3.49 (t, 2H), 3.11-3.10 (br, 2H), 2.63-2.58 (m, 2H), 1.86-1.84 (m, 1H), 1.68-1.65 (m, 1H), 1.60-1.55 (m, 2H). MS (ESI) 596.36 (M⁺+H).

### EXAMPLE 24

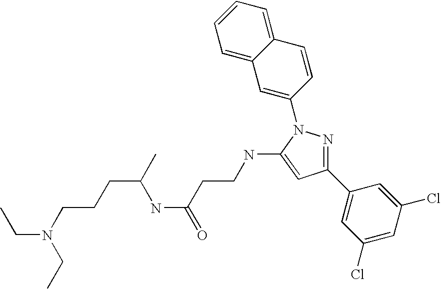

A similar procedure to example 23 was followed using 2-amino-5-diethylaminopentae to give N³-[3-(3,5-dichlorophenyl)-1-(2-naphthyl)-1H-pyrazol-5-yl]-N¹-[4-(diethylamino)-1-methylbutyl]-β-al aninamide. MS 609.20 (M⁺+H).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. Therefore, the invention is defined by the claims which follow and not limited by the examples.

1. A compound represented by Formula (I): 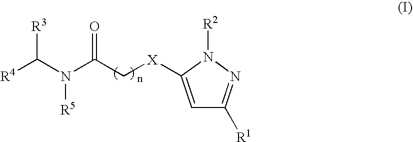
wherein:
R¹ and R² are each independently selected from —(C₀₋₆alkyl)-W—(C₆₋₁₄aryl), —(C₀₋₆alkyl)-W-heterocycle and —(C₀₋₆alkyl)-W—(C₃₋₇cycloalkyl);
R³ and R⁴ are each independently selected from —C₀₋₆alkyl, —(C₀₋₆alkyl)-W—(C₁₋₆alkyl), —(C₀₋₆alkyl)-W—(C₃₋₇cycloalkyl), —(C₀₋₆alkyl)-W—(C₆₋₁₄aryl), —(C₀₋₆alkyl)-W-heterocycle, and —C(O)OR⁶;
where each of R¹, R², R³ and R⁴ is independently unsubstituted or substituted with 1-7 substituents, where each of said 1-7 substituents is independently selected from halo, hydroxy, —O—C₁₋₃alkyl, trifluoromethyl, —C₁₋₃alkyl, —CO₂R⁶, —CN, —N(R⁶)₂, —NR⁶COR⁶, —NRSO₂R⁶, and —CONR⁶;
R⁵ is selected from hydrogen, —C₀₋₆alkyl, —(C₀₋₆alkyl)—(C₆₋₁₄aryl), —(C₀₋₆alkyl)-heterocycle, —(C₀₋₆alkyl)-C₃₋₇cycloalkyl and —(C₀₋₆alkyl)-CO₂R⁶;
R⁶ is independently selected from C₁₋₆alkyl and NR⁵C(N)NH₂, or two R⁶ join to form a ring selected from pyrrolidinyl, piperidinyl and azepanyl;
X is CH₂, N, O or S;
W is selected from a single bond, —O—, —S—, —SO—, —SO₂—, —CO—, —CO₂—, —CONR⁶— and —NR⁶—; and
n is 0-6;
or a pharmaceutically acceptable salt or an individual diastereomer thereof.

2. The compound of claim 1, wherein:
R¹ and R² are each independently selected from —(C₆₋₁₄aryl) and —(C₆₋₁₄heteroaryl);
R³ and R⁴ are each independently selected from —C₀₋₆alkyl, —(C₀₋₆alkyl)—(C₆₋₁₄aryl), —(C₀₋₆alkyl)-(C₆₋₁₄heteroaryl),
where each of R¹, R², R³ and R⁴ is independently unsubstituted or substituted with 1-7 substituents, where each of said 1-7 substituents is independently selected from halo, hydroxy, —O—C₁₋₃alkyl, trifluoromethyl, —C₁₋₃alkyl, —CO₂R⁶, —CN, —N(R⁶)₂, —NR⁶COR⁶, —NRSO₂R⁶, and —CONR⁶;
R⁵ is —C₀₋₆alkyl;
R⁶ is independently selected from C₁₋₆alkyl and NR⁵C(N)NH₂, or two R⁶ join to form a ring selected from pyrrolidinyl, piperidinyl and azepanyl;
X is CH₂ or O;
W is selected from a single bond, —O—, —S—, —SO—, —SO₂—, —CO—, —CO₂—, —CONR⁶— and —NR⁶—; and
n is 0-6;
or a pharmaceutically acceptable salt thereof, or an individual diastereomer thereof.

3. A compound selected from the group consisting of: 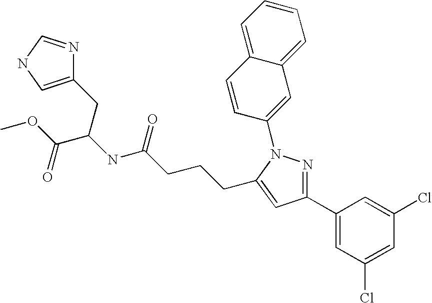 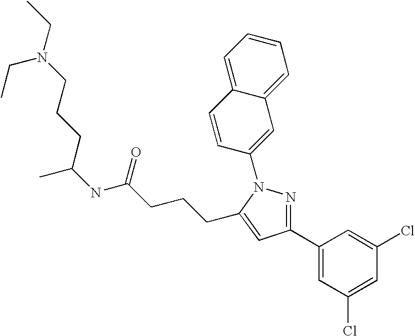 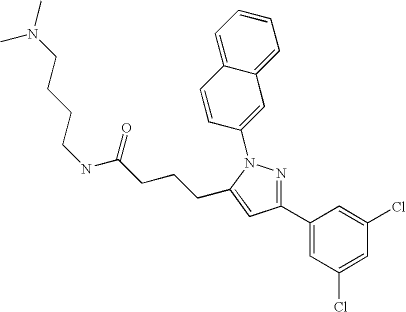 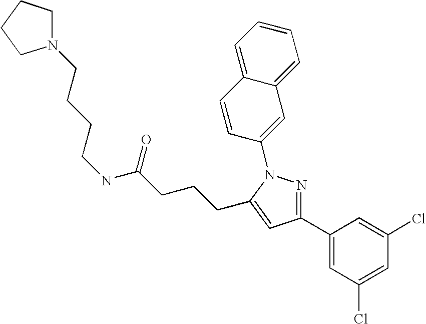 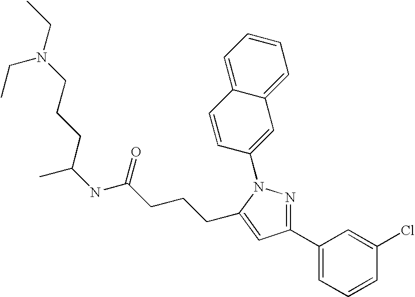 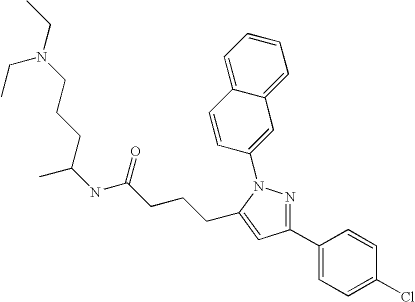 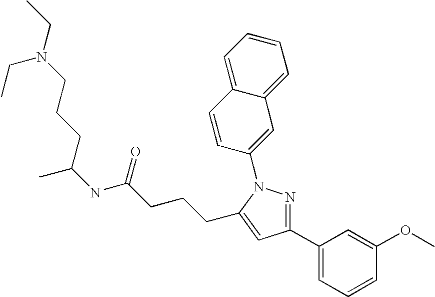 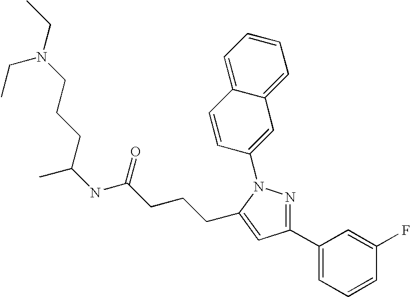 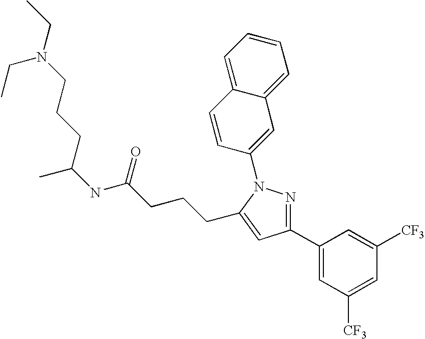 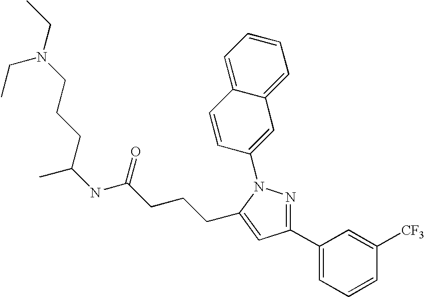 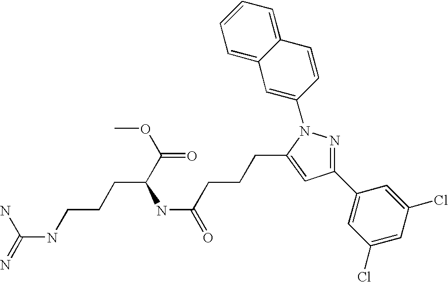 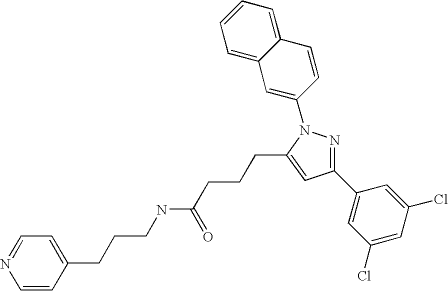 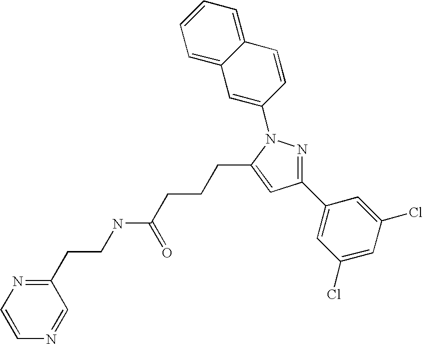 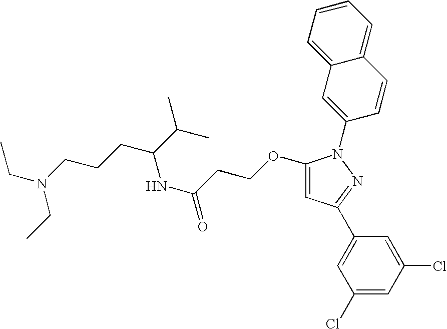 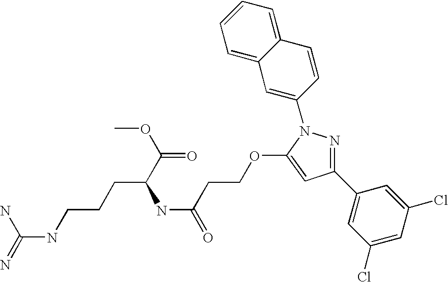 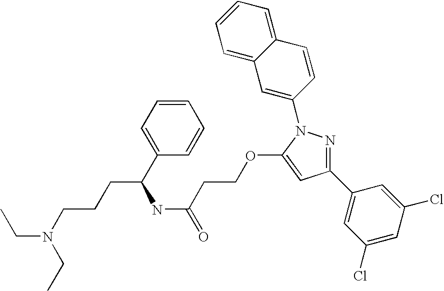 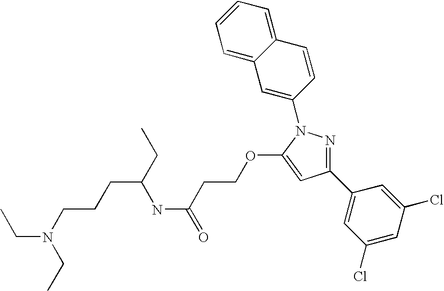 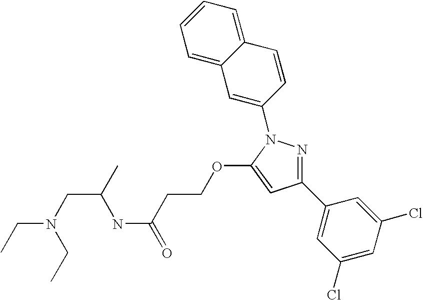 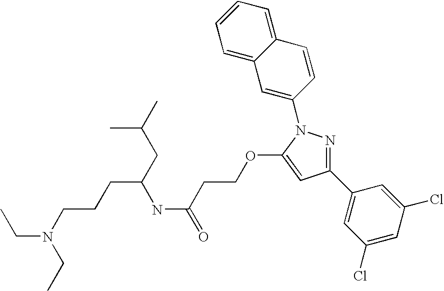 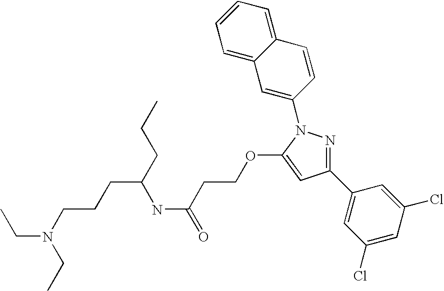 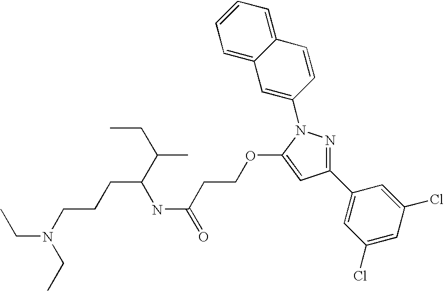 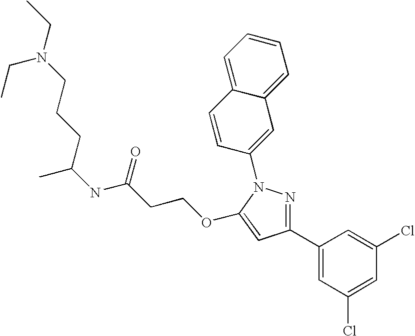 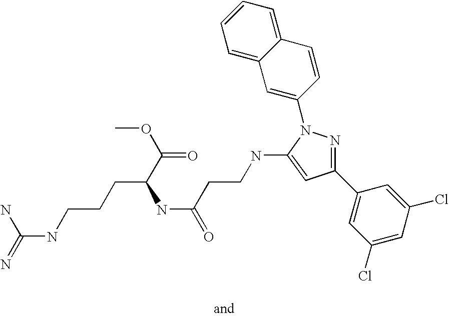 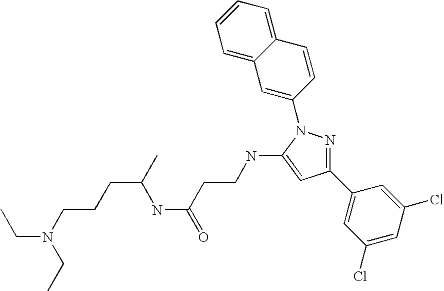
or a pharmaceutically acceptable salt or individual diastereomer thereof.

4. A pharmaceutical composition which comprises an inert carrier and the compound of claim 1, or a pharmaceutically acceptable salt or individual diastereomer thereof.

5. A method for modulation of chemokine receptor activity in a mammal which comprises the administration of an effective amount of the compound of claim 1, or a pharmaceutically acceptable salt or individual diastereomer thereof.

6. A method for treating, ameliorating, controlling or reducing the risk of an inflammatory or immunoregulatory disorder or disease which comprises the administration to a patient of an effective amount of the compound of claim 1, or a pharmaceutically acceptable salt or individual diastereomer thereof.

7. The method according to claim 6, wherein said disorder or disease is selected from the group consisting of multiple sclerosis, rheumatoid arthritis, atherosclerosis, chronic obstructive pulmonary disease, obesity, type II diabetes and metabolic syndrome.
